(19) United States
(12) Patent Application Publication
(10) Pub. No.: US 2009/0324443 A1
(43) Pub. Date: Dec. 31, 2009

(54) GAS TREATMENT

(76) Inventors: John Christopher Whitehead, Cheshire (GB); Kui Zhang, Manchester (GB); David John Glover, Cheshire (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/278,319

(22) PCT Filed: Feb. 19, 2007
(86) PCT No.: PCT/GB07/00551

§ 371 Date: Aug. 5, 2008

(30) Foreign Application Priority Data
Feb. 17, 2006 (GB) 0603235.3
Jun. 21, 2006 (GB) 0612249.3

(51) Int. Cl. A61L 9/16; B01D 53/38; B01J 19/08

(52) U.S. Cl. 422/4; 422/171

(57) ABSTRACT

In one aspect, the invention provides a gas treatment apparatus (1) comprising a gas-flow path and a plurality of reactor units (5)-(7) through which gas to be treated may flow arranged in series along the path. The reactor units (5)-(7) are adapted to generate a non-equilibrium plasma. This aspect of the invention may be used for decomposing pollutant materials in a gas (e.g. air). When air is being treated, the apparatus of this aspect of the invention is advantageously provided, downstream of the final reactor unit in series, with at least one catalyst bed (8) incorporating a catalyst capable of decomposing ozone. A further aspect of the invention provides apparatus (1) for decomposing a pollutant material dispersed in a gas, the apparatus comprising a gas flow path along which are provided for gas flow therethrough (i) at least one reactor unit (5) which is adapted to generate a non-equilibrium plasma and produce ozone in the gas, and (ii) downstream of (i), at least one catalyst bed (8) incorporating a catalyst capable of decomposing ozone.

The present invention relates to an apparatus and method for treating a gas. The invention embraces a method and apparatus for decomposing pollutant materials dispersed in gases including but not limited to air, nitrogen, argon and xenon. The pollutant may, for example, be Volatile Organic Compounds (VOCs), biological agents and other hazardous air pollutants (HAPs). The invention relates particularly (but not necessarily exclusively) to the treatment of waste gas streams. The invention also relates to a method of treating air to produce ozone therefrom.

There are a number of applications where treatment of a gas is required. One such example is the treatment of air (e.g. waste gas streams) incorporating air borne contaminants such as gaseous phase organic compounds (e.g. Volatile Organic Compounds (VOCs)). A further example is the treatment of nitrogen, argon or xenon incorporating VOCs.

VOCs are contaminants found across a range of market sectors from semi-conductor manufacturing plants to chemical processing plants including paint, coatings and chemical manufacturing. The use of VOCs in industrial processes is widespread and it is important to remove these contaminants from air which is either to be recirculated or released into the environment. Adsorption methods such as activated carbon are widely used to remove VOCs from air but there are a wide range of VOCs and the absorption efficiency of carbon is varied. Whilst carbon is a solution for many VOCs, compounds like acetic acid are not absorbed efficiently so that a large volume of carbon is required for efficient removal. This is expensive, requires significant energy to push the air through the system and there are disposal costs to be taken into account. Thermal catalysis is also widely used for the removal of pollutants from waste gases but expensive catalysts containing precious metals are often required together with high energy input to obtain the necessary operating temperatures. Another issue is the lifetime of the catalysts where poisoning by some pollutants is a problem.

Lubrication oil mists, oil fumes and emulsion mists are produced during various industrial processes including metal cutting, rolling and hardening etc. where the oil is used as a lubricant, coolant or hydraulic fluid. The use of lubrication oils in industrial processes is widespread and it is important to remove these contaminants from air which is either to be recirculated or released into the environment. Oil mists, which are fine particles, may be removed by HEPA-based filter (high efficiency paper filters) systems but these systems are unable to remove the molecular oil vapour component. There is currently no system on the market which can effectively remove oil vapours from polluted air. Carbon might be thought to be a possible choice but a large volume of material is required causing high back pressures and hence high energy requirements associated with this large volume and there are disposal costs associated with the spent hazardous waste.

There have been a number of proposals based on plasma reactions for treatment of air. It is generally understood that the completeness of plasma reactions is principally a function of the input power density for a given residence time and as a consequence there is wide use of the normalized energy density unit ‘Joules per litre’ to compare the efficiency and activity of different plasma reactors. For example see article SAE 982508-BM Penetrante et al.

Possibly the most important consideration in commercialisation of plasma system design is the energy efficiency as it relates directly to both running cost and capital cost of power supplies. In addition, certain applications such as vehicle exhaust aftertreatment have additional constraints due to finite on-board power availability. As a consequence, several approaches have been investigated in an attempt to improve plasma processing efficiency beyond the apparently fixed constraints dictated by the energy density considerations.

Plasma assisted catalysis (for example see EP 1274504 B1 and comprehensive references therein) employs a catalyst stage downstream of a plasma. It has been suggested that this approach works by using the plasma to produce activated or partially oxidized hydrocarbons which flow downstream and improve the efficiency of certain catalysts, particularly at low temperatures. This approach is particularly applicable to improving the low temperature processing efficiency of internal combustion engine exhaust gases under lean conditions, but is very dependent on the catalyst design—the surface must be carefully designed to benefit from the plasma—and is not widely applicable to industrial gas processing, where for example mixed contaminant streams and variable process conditions often damage catalysts.

Plasma assisted trapping or adsorption (for example WO 01/30485 A1) describes changing the residence time of selected species in a plasma reactor in order to break the link between joules per litre input power and reactive species. The device described is again applicable to the treatment of species derived from the exhausts of internal combustion engines and particularly applicable to processing trapped soot. This approach is not generally applicable to industrial gas processing as many species required to be processed cannot be easily trapped or adsorbed.

While there are references to serial operation of plasmas (such as US 2004/0134890 A1) these suggest (for example P2 line 3 of US 2004/0134890 A1) that series operation will improve residence time effect implying that the impact will be approximately additive. This is supported by the observation that the energy density to achieve 63% (1/e) decomposition of toluene falls from 396 joules per litre with one plasma torch to 173 joules per litre with three plasma torches—a factor of 2.3.

A further application for air treatment relates to the production of ozone from air, e.g. to provide an environment relatively enriched in ozone for hygienic purposes. However many devices for producing ozone from air also result in the production of relatively high levels of NO_{X} gases (i.e. NO and NO₂ although it should be noted that N₂O is not normally considered a component of NO_{X}—see R. P. Wayne, Chemistry of Atmospheres, 3^{rd} ed. OUP, 2000, p 166). For these reasons, ozone tends to be produced from pure oxygen rather than from air.

WO-A-0014010 (The Victoria University of Manchester) discloses an air purification device comprising two electrodes having a dielectric material (e.g. glass beads or alumina) therebetween and means for applying a potential difference across the electrodes. The electrodes are air-permeable and the dielectric material is in the form of an air-permeable, fixed bed. The apparatus further incorporates means (e.g. a fan) to provide airflow through one electrode, across the fixed bed of dielectric material and through the other electrode. In use, AC electric power at high voltage is applied between the two electrodes

WO-A-0014010 proposes use of the device for reducing the level of airborne particulates such as smoke, dust, soot, aerosols and bacteria and it is in such applications that the apparatus is currently being commercialised. Not only are such particles removed from the air but they are also “burnt-off” on the bed so there are no remaining deposits. There is however no disclosure in WO-A-0014010 as to the use of the device for the removal of gaseous organic compounds (e.g. VOCs and HAPs).

WO-A-0014010 does disclose that operation of the apparatus described therein leads to the production of ozone although the levels achieved are insufficient for some commercial ozone generation applications.

According to a first aspect of the present invention there is provided gas treatment apparatus comprising a gas flow path and a plurality of reactor units through which gas may flow arranged in series along said path, said reactor units being adapted to generate a non-equilibrium plasma.

According to a second aspect of the present invention there is provided a method of treating a gas containing oxygen comprising passing the gas in series through a plurality of reactor units in which a non-equilibrium plasma is generated.

The method of the second aspect of the invention is particularly effective for the treatment of air since this provides a source of oxygen for conversion by the non-equilibrium plasma to ozone which we believe to be an important feature of the method (see below). If however the gas to be treated does not incorporate oxygen (or only insufficient oxygen) then it is possible to effect the method of the second aspect of the invention by introducing oxygen (or a source thereof) into the gas upstream of the non-equilibrium plasma to effect the production of ozone.

We have established, and this forms the basis of the first and second aspects of the present invention, that an arrangement of serially arranged reactor units in which a non-equilibrium (non-thermal) plasma is generated is very effective for the treatment of a gas containing oxygen (e.g. air) for a variety of applications. These include treatment of the gas to decompose gas-borne contaminants, e.g. organic compounds and biological agents. This result is particularly surprising in the light of the fact that we have established that the use of a single reactor unit generating a non-equilibrium plasma produces little or no decomposition of gas-borne (particularly airborne) contaminants. Put another way, a single reactor unit has been found to be virtually ineffective for the treatment of airborne contaminants whereas a plurality of such cells arranged in series is highly effective.

Preferably there are at least three of the reactor units arranged in series.

By way of illustration, we have established that an arrangement of three of the reactor units in series is able to remove 72% of toluene from an air stream where the percentage removal achieved by a single unit is less than 0.1%. Normally, this magnitude of destruction would only be achievable using a much larger plasma system consuming much greater energy; the improvement in energy efficiency is a key feature of the invention. Another aspect of prior art high energy plasma systems operating in air gas streams is that large amounts of NOx are also produced as unwanted by-products in addition to the desired destruction of the pollutant. In contrast, an arrangement of the present invention produces only low NOx levels. Thus the invention provides a unique combination of high destruction levels coupled with minimal generation of NOx at lowered energy consumption rates (<50 W).

We have shown for the first time that the passage of air in series through a plurality of reactor units in which a non-equilibrium plasma is generated can improve process energy efficiency significantly above that which would be expected by an additive effect of increased residence time. Rather than a simple additive effect the energy density required to achieve 63% (1/e) decomposition of toluene decreases by a factor of 400 by changing from single cell operation to 3 cells in series. This has significant implications in plasma gas processing, allowing very low power operation of multiple cells to achieve process efficiencies that have previously only been observed with high input energy plasma devices.

Also by way of illustration we have established that an arrangement of three of the reactor units in series shows increased energy efficiency when scaled up to commercially viable air flows. The arrangement is able to remove 100% of 25 ppm toluene from air for energy densities of less than or equal to 18.J/L at a face velocity of 0.4 m/s.

We do not wish to be bound by theory, but believe that the synergistic effect that is achieved by combining two or more of the reactor units in series has its origin in the generation (in the non-equilibrium plasma of an upstream cell) of activated species for example excited states, radicals, ions and long lived intermediates such as ozone which impart increased efficiency to the downstream cells. We believe that the arrangement of the units optimises the production of key intermediates for particular input energies.

Additionally, the series arrangement of the reactor units with passage of air therethrough produces significant amounts of ozone without undesirable levels of NOₓ gases.

According to a third aspect of the present invention there is provided a method of treating a gas containing oxygen to remove gas-borne contaminants therefrom, the method comprising passing the gas to be treated in series through a plurality of reactor units in which a non-equilibrium plasma is generated. The gas to be treated may be air.

According to a fourth aspect of the present invention, there is provided a method of generating ozone comprising passing air in series through a plurality of reactor units in which a non-equilibrium plasma is generated.

The reactor units employed in the first to fourth aspects of the invention may each be reactor cells which comprise:

(i) a pair of spaced, air-permeable electrodes,

(ii) an air-permeable fixed bed of a dielectric material extending between the electrodes; and

(iii) means for applying a potential difference across the electrodes to provide a non-equilibrium plasma in the bed between the electrodes,

said cells being arranged such that the gas flow path is through the electrodes and the fixed beds.

According to a fifth aspect of the present invention there is provided apparatus for treating a gas (eg air) to remove gaseous phase organic pollutants contained therein, the apparatus comprising a gas flow path, a plurality of reactor cells arranged in series along said path, and means for causing the gas to flow along said path and through the reactor cells, wherein the reactor cells comprise:

(i) a pair of spaced, air-permeable electrodes,

(ii) an air-permeable fixed bed of a dielectric material extending between the electrodes; and

(iii) means for applying a potential difference across the electrodes to provide an electric field in the bed between the electrodes,

said cells being arranged such that the gas flow path is through the electrodes and the fixed beds.

According to a sixth aspect of the present invention there is provided a method of treating a gas (eg air) comprising passing the gas to be treated in series through a plurality of reactor cells each comprising:

(i) a pair of spaced, air-permeable electrodes, and

(ii) an air-permeable fixed bed of a dielectric material extending between the electrodes,

said gas passing through the electrodes and the fixed beds, said method further comprising applying a potential difference across the electrodes of each reactor cell to provide an electric field between the electrodes.

The term “fixed bed” is intended to mean that the dielectric material (which extends between the electrodes) does not move in normal usage of the device. The term is intended to cover inter alia a bed of discrete particles, a foam, a sponge-like structure and a bed of elongate elements such as filaments arranged in contacting relationship with air gaps therebetween. Most preferably the bed is comprised of discrete bodies (e.g. beads) in contacting relationship. Preferred embodiments of “fixed bed” for use in accordance with the invention may be characterised as “packed-beds”.

Preferably there are at least three of the reactor cells arranged in series.

In preferred embodiments of the apparatus, each reactor cell may comprise several sub-sections arranged across the gas flow path at equal and opposite angles to each other (i.e. somewhat of “zig-zag” configuration). This increases the cross-sectional area of a reactor cell for a given cross-section of gas flow path.

The reactor cells (particularly those employed for the fifth and sixth aspects of the invention) will generally have an overall thickness (i.e. the distance between the outer surfaces of the two electrodes) which is significantly less than either of their other two dimensions. The cells, may for example be square, rectangular or circular in plan view (i.e. as seen looking towards one of the electrodes) although other configurations are possible.

The electrodes may be formed of a metal gauze or mesh or other conductive porous materials. Suitable materials include copper, steel, nickel and reticulated vitreous carbon.

A wide range of dielectric materials may be used but most preferably the material has a dielectric constant less than 100. More preferably less than 50 and even more preferably less than 25. Typically but not exclusively the dielectric material used in the reactor cells has a dielectric constant of less than 20. The use of a material with a reasonably low dielectric constant, such as glass (which is the preferred dielectric material for use in the invention), allows cost savings over dielectric materials having a higher dielectric constant. In addition the use of these materials minimises or eliminates the production of unwanted species such as oxides of nitrogen, NOx. Silica, alumina, or other suitable dielectric (zirconia, sapphire, etc.) could be used in place of glass. It is however possible to use materials with higher dielectric constants, e.g. up to 1000 or above, although higher levels of NOx will be generated. One of example of material having a high dielectric constant that may be used is barium titanite.

Preferably the air permeable bed is comprised of discrete bodies of dielectric material in contacting relationship. The discrete bodes are preferably particles and preferably regularly shaped particles. Even more preferably, the particles are at least generally spherical and are most preferably in the form of beads. The diameter of the beads is preferably about 1 mm to 12 mm, more preferably 2 to 10 mm even more preferably 4-8 mm. A diameter of about 6 mm is particularly suitable. Glass in the form of wool, chips, or extruded foam could be used in place of beads provided that air permeability is retained and that elements of the dielectric material are in a contacting relationship, although regularly spaced beads give an advantage in that better airflow is allowed through the dielectric bed.

The potential difference applied across the electrodes should be an AC voltage, e.g. greater than 1 kVₚₖ₋ₚₖ. For the purpose of this invention, and AC voltage is defined as an oscillating wave including but not limited to sine waves, pseudo-sine waves, square waves, saw toothed waves and pulsed DC. The voltage may for example be 1-100 kVₚₖ₋ₚₖ. The frequency may be 10-100 kHz, although voltage such as mains at 50 Hz or 60 Hz could be used.

The reactor cells may be of the type disclosed in WO-A-0014010.

The present invention will find use in the treatment of air to remove various organic pollutants, e.g. hydrocarbons and halogenated solvents (e.g. methylene chloride, carbon tetrachloride and trichloroethylene. It is envisaged that the present invention will be particularly useful for the removal of pollutants such as VOCs, HAPs and oil vapour from gas streams. Additional applications include the removal of nanoparticulates, oil mists, odours and biological agents from air. Specific further applications include treatment of air in an aircraft cabin and vehicle exhaust aftertreatment.

The method and apparatus in accordance with the invention may be used in conjunction with UV, catalysts and/or filters depending on the particular processes concerned.

The present invention also finds use in the production of ozone from air with low levels of NOₓ.

In an advantageous development of the first to sixth aspects of the present invention there is provided downstream of the last reactor unit in series a catalyst bed incorporating a catalyst capable of decomposing ozone. This embodiment is particularly effective for those of the first to sixth aspects of the invention which relate to the treatment of waste gas streams containing organic contaminants since we have surprisingly found that the ozone decomposition catalyst is able to effect further decomposition of contaminants which survive passage through the reactor units.

The ozone decomposition catalyst is preferably manganese dioxide.

The catalyst capable of decomposing ozone may be a supported catalyst. Thus, for example, the catalyst bed may comprise a honeycomb (e.g. metal or cordierite) coated with the manganese dioxide.

In the method of the invention, for decomposing an organic material or other pollutant, the ozone decomposition catalyst will generally result in the production of both carbon dioxide and carbon monoxide from the organic material. In order to reduce the carbon monoxide level, a catalyst (e.g. copper oxide) capable of decomposing carbon monoxide is preferably employed in conjunction with the ozone decomposition catalyst. In a preferred embodiment of the invention, the carbon monoxide decomposition catalyst is provided in a catalyst unit provided downstream (preferably immediately downstream) of the catalyst unit incorporating the ozone decomposition catalyst. However we do not preclude the possibility of the ozone and carbon monoxide decomposition catalysts being used either as an admixture or impregnated on a common support.

The use of an ozone decomposition catalyst is an important aspect of the present invention in its own right and therefore according to a seventh aspect of the present invention there is provided apparatus for decomposing a pollutant material dispersed in a gas, the apparatus comprising a gas flow path along which are provided for gas flow therethrough.

(i) at least one reactor unit which is adapted to generate a non-equilibrium plasma and produce ozone in the gas, and

(ii) downstream of (i), at least one catalyst bed incorporating a catalyst capable of decomposing ozone.

According to a an eighth aspect of the present invention there is provided a method of decomposing a pollutant material dispersed in the gas phase, the method comprising subjecting oxygen with which the pollutant material is, or is to be, admixed to a non-equilibrium plasma which is adapted to generate ozone, and contacting the plasma treated oxygen containing dispersed pollutant material with a catalyst capable of decomposing ozone.

We have established, and this forms the basis of the seventh and eighth aspects present invention, that the combined use of a non-equilibrium plasma (configured in such a way as to generate ozone in a gas) and an ozone decomposition catalyst is surprisingly effective for decomposing pollutant materials (particularly organic materials, e.g. VOCs) dispersed in a gas. Such a configuration shows vastly improved power efficiencies when compared to the use of plasma alone to such an extent that it is possible to remove 100% of the pollutant when employing a catalyst where otherwise only a small reduction of the pollutant could be achieved. Thus, by way of illustration Example 6 below which relates to the destruction of toluene (and which was conducted under different conditions from Example 2) uses low power conditions which result in destruction of 100% toluene by using the apparatus/method in accordance with the seventh and eighth aspects of the invention but only 36% destruction when the catalyst is not employed. Additionally the procedure disclosed in Example 7 which was conducted using lower power conditions and lower flow rates than employed in Example 6 resulted in 100% destruction of cyclohexane using an apparatus/method in accordance with the invention but only about 24% destruction without the catalyst.

The seventh and eighth aspects of the invention are particularly effective for the case where the pollutant material is dispersed in the gas which is subjected to the non-equilibrium plasma. Thus in this case the invention may be applied to the treatment of polluted gas which is firstly subjected to the non-equilibrium plasma and then contacted with the catalyst capable of decomposing ozone.

Therefore in accordance with a ninth aspect of the present invention there is provided a method of treating gas containing pollutant dispersed in the gas comprising passing the gas and the pollutant through at least one reactor unit in which a non-equilibrium plasma is generated with production of ozone and through a catalyst unit located downstream of the reactor unit(s) incorporating a catalyst capable of decomposing ozone.

In this ninth aspect of the invention, the non-equilibrium plasma will-decompose a certain amount of the pollutant and further decomposition thereof will be effected once the gas is contacted with the catalyst capable of decomposing ozone.

The method of the ninth aspect of the invention is particularly effective for the treatment of waste air streams (containing airborne pollutant) since in this case the air provides a source of oxygen for conversion by the non-equilibrium plasma to ozone. If however the gas to be treated does not incorporate oxygen (or only insufficient oxygen) then it is possible to effect the method of the third aspect of the invention by introducing oxygen (or a source thereof into the waste gas stream upstream of the non-equilibrium plasma to effect the production of ozone.

Although it is preferred that the gas containing the pollutant is passed through the reactor unit (which generates the non-equilibrium plasma), the seventh to ninth aspects of the invention are however also effective for the case where air or oxygen (not containing the dispersed pollutant) is subjected to a non-equilibrium plasma, the pollutant is then dispersed in the plasma treated gas air (or oxygen), and the mixture of dispersed pollutants and plasma treated air (or oxygen) is contacted With the ozone decomposition catalyst.

The catalyst capable of decomposing ozone seventh to ninth aspects of the invention may, for example, comprise magnesium dioxide, which is particularly advantageous because it is effective for ozone decomposition at ambient temperature. Thus, with the use of manganese dioxide as the ozone decomposition catalyst, the method in accordance with eighth and ninth aspects of the invention may advantageously be effected at ambient temperature thereby avoiding any need to heat the incoming air stream. However other ozone decomposition catalysts may be used.

The catalyst capable of decomposing ozone may be a supported catalyst. Thus, for example, the catalyst bed may comprise a honeycomb (e.g. metal or cordierite) coated with the manganese dioxide.

In the method of the eighth and ninth aspects of the invention, for decomposing an organic material or other pollutant, the ozone decomposition catalyst will generally result in the production of both carbon dioxide and carbon monoxide from the organic material. In order to reduce the carbon monoxide level, a catalyst (e.g. copper oxide) capable of decomposing carbon monoxide is preferably employed in conjunction with the ozone decomposition catalyst. In a preferred embodiment of the invention, the carbon monoxide decomposition catalyst is provided in a catalyst unit provided downstream (preferably immediately downstream) of the catalyst unit incorporating the ozone decomposition catalyst. However we do not preclude the possibility of the ozone and carbon monoxide decomposition catalysts being used either as an admixture or impregnated on a common support.

For the purposes of the seventh to ninth aspects of the invention there are preferably at least three of the reactor units (each capable of generating a non-equilibrium plasma) arranged in series.

The reactor units may each be reactor cells which comprise:

(i) a pair of spaced, gas-permeable electrodes,

(ii) an gas-permeable fixed bed of a dielectric material extending between the electrodes; and

(iii) means for applying a potential difference across the electrodes to provide a non-equilibrium plasma in the bed between the electrodes, said cells being arranged such that the gas flow path is through the electrodes and the fixed beds.

The term “fixed bed” is intended to mean that the dielectric material (which extends between the electrodes) does not move in normal usage of the device. The term is intended to cover inter alia a bed of discrete particles, a foam, a sponge-like structure and abed of elongate elements such as filaments arranged in contacting relationship with air gaps therebetween. Most preferably the bed is comprised of discrete bodies (e.g. beads) in contacting relationship. Preferred embodiments of “fixed bed” for use in accordance with the invention may be characterised as “packed-beds”.

In preferred embodiments of the apparatus, each reactor cell may comprise several sub-sections arranged across the gas flow path at equal and opposite angles to each other (i.e. somewhat of “zig-zag” configuration). This increases the cross-sectional area of a reactor cell for a given cross-section of gas flow path.

The reactor cells will generally have an overall thickness (i.e. the distance between the outer surfaces of the two electrodes) which is significantly less than either of their other two dimensions. The cells, may for example be square, rectangular or circular in plan view (i.e. as seen looking towards one of the electrodes) although other configurations are possible.

The electrodes may be formed of a metal gauze or mesh or other conductive porous materials. Suitable materials include copper, stainless steel, nickel and reticulated vitreous carbon.

A wide range of dielectric materials may be used but most preferably the material has a dielectric constant less than 100, more preferably less than 50 and even more preferably less than 25. Typically but not exclusively the dielectric material used in the reactor cells has a dielectric constant of less than 20. The use of a material with a reasonably low dielectric constant, such as glass (which is the preferred dielectric material for use in the invention), allows cost savings over dielectric materials having a higher dielectric constant. In addition the use of these materials minimises or eliminates the production of unwanted species such as oxides of nitrogen, NOx. Silica, alumina, or other suitable dielectric (zirconia, sapphire, etc.) could be used in place of glass. It is however possible to use materials with higher dielectric constants, e.g. up to 1000 or above, although higher levels of NO_{X} will be generated. One example of material having a high dielectric constant that may be used is barium titanite.

Preferably the gas permeable bed is comprised of discrete bodies of dielectric material in contacting relationship. The discrete bodes are preferably particles and preferably regularly shaped particles. Even more preferably, the particles are at least generally spherical and are most preferably in the form of beads. The diameter of the beads is preferably about 1 mm to 12 mm, more preferably 2 to 10 mm even more preferably 4-8 mm. A diameter of about 6 mm is particularly suitable. Glass in the form of wool, chips, or extruded foam could be used in place of beads provided that gas permeability is retained and that elements of the dielectric material are in a contacting relationship, although regularly spaced beads give an advantage in that better gas flow is allowed through the dielectric bed.

The potential difference applied across the electrodes should be an AC voltage, e.g. greater than 1 kVₚₖ₋ₚₖ. For the purpose of this invention, and AC voltage is defined as an oscillating wave including but not limited to sine waves, pseudo-sine waves, square waves, saw toothed waves and pulsed DC. The voltage may for example be 1-100 kVₚₖ₋ₚₖ. The frequency may be 10-100 kHz, although voltage such as mains at 50 Hz or 60 Hz could be used.

The reactor cells may be of the type disclosed in WO-A-0014010.

Although the use of “fixed bed” (also known as “packed bed”) reactors as described above is a preferred embodiment of the invention, it is also possible to use non-equilibrium plasma reactors of different designs. Thus, for example, in situations where the gas contains highly conductive materials such as carbon-based particulates or water vapour then it may be preferable to use one or more non-equilibrium plasma reactors of a dielectric barrier design.

The present invention will find use in the treatment of gases including but not limited to air, nitrogen, argon and xenon to remove various organic pollutants, e.g. hydrocarbons and halogenated solvents (e.g. methylene chloride, carbon tetrachloride and trichloroethylene). It is envisaged that the present invention will be particularly useful for the removal of pollutants such as VOCs, HAPs and oil vapour from gas streams. Additional applications include the removal of nanoparticulates, oil mists, odours and biological agents from air. Specific further applications may include but not limited to treatment of air in an aircraft, automobile and submarine cabin and vehicle exhaust aftertreatment.

The method and apparatus in accordance with the invention may be used in conjunction with UV, catalysts and/or filters depending on the particular processes concerned.

The invention will be further described by way of example only with reference to the accompanying drawings, in which:

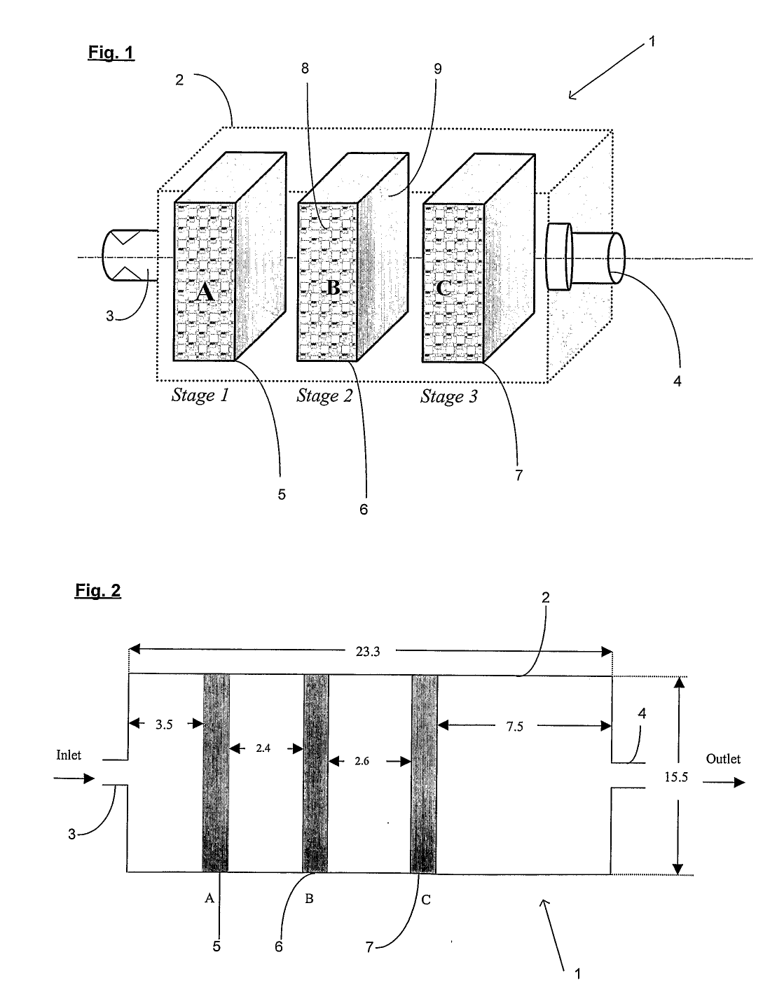

FIG. 1 schematically illustrates one embodiment of apparatus in accordance with the invention;

FIG. 2 schematically illustrates an embodiment of apparatus in accordance with the invention employed in the experimental procedure of Examples 1-3;

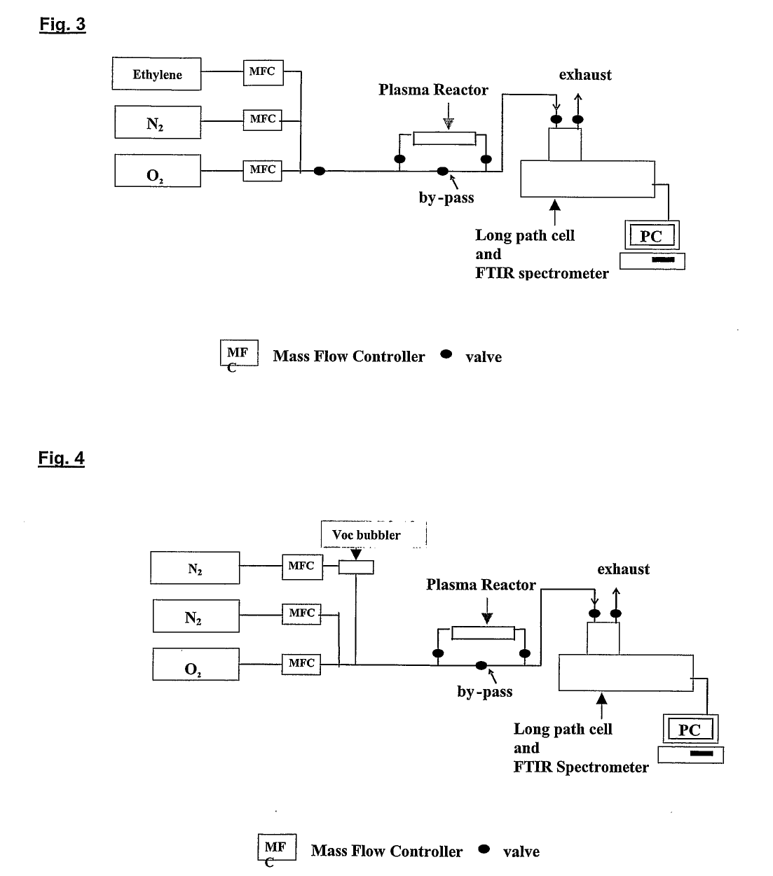

FIG. 3 illustrates the experimental set-up employed in Example 1;

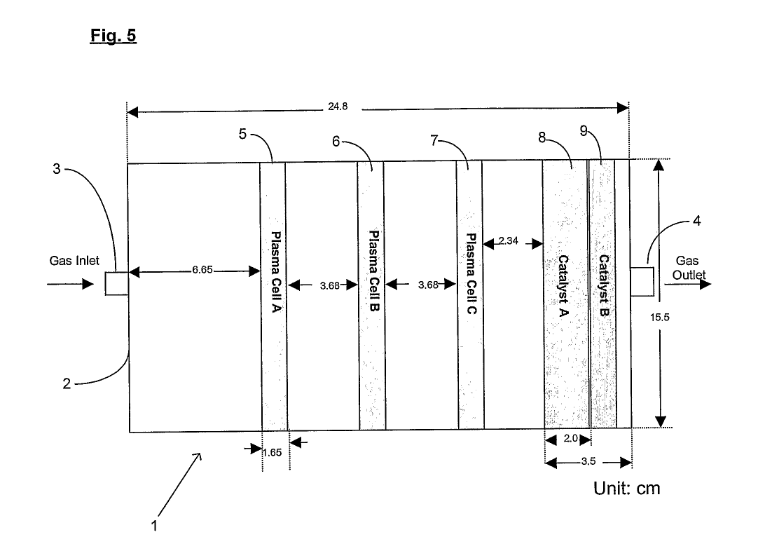

FIG. 4 illustrates the experimental set-up employed in Example 2;

FIG. 5 illustrates the apparatus employed in Examples 6 and 7;

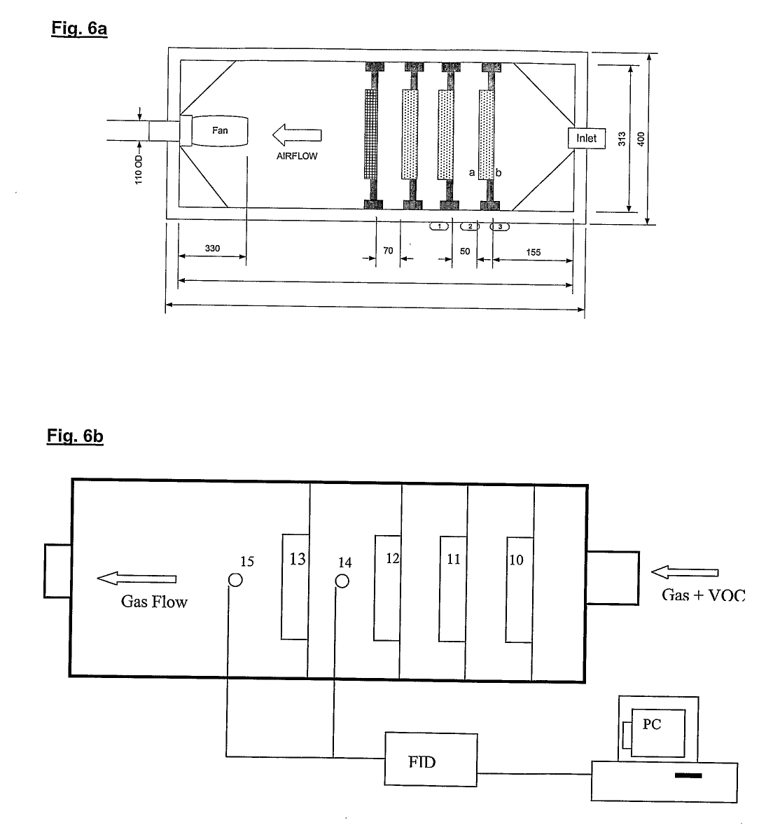

FIGS. 6a and 6b illustrate the apparatus employed in Example 8 (and, in a modified form, in Examples 4 and 5);

FIG. 7 illustrates the results of Example 5; and

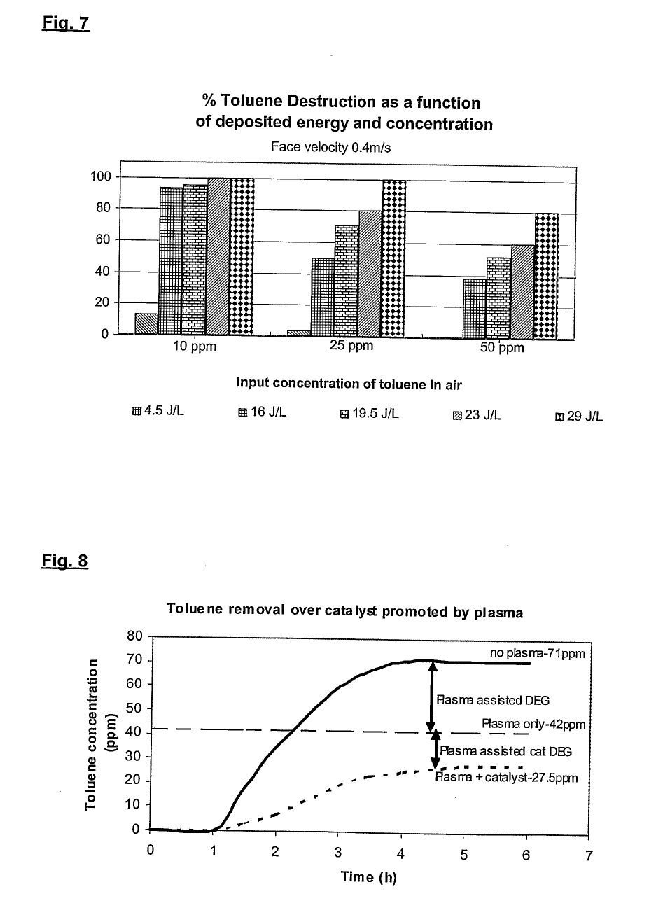

FIG. 8 illustrates the results of Example 8

The apparatus 1 illustrated in FIG. 1 comprises a housing 2 formed with an inlet 3 and an outlet 4. Located in series within the housing 2 are three reactor cells 5-7 positioned such that gas entering the apparatus 1 through inlet 3 has to flow through each of cells 5-7 before reaching outlet 4.

The units 5-7 are identical with each other and comprise an air-permeable bed of packed glass spheres 8 (e.g. 6 mm diameter) sandwiched between two air-permeable electrodes 9.

The apparatus further comprises three separate AC power supplies (not shown) each associated with a respective one of the units 5-7 and also means (not shown) such as a fan or the like for moving air through the apparatus from inlet 3 to outlet 4 via cells 5-7.

In use of the apparatus 1, the power supplies are used to apply high voltage, high frequency energy across the electrodes 9 of each cell 5-7. Air to be treated enters apparatus 1 via inlet 3 and passes in series through units 5-7 prior to exiting housing 2 via outlet 4.

Reference is now made to FIG. 2 which provides more specific details of the embodiment of apparatus employed in Examples 1-3 below.

The apparatus of FIG. 2 (for which all dimensions are in centimetres) comprises three plasma reactors in series contained in a gas-tight box with an external plasma power supply. For convenience, the same reference numerals employed in FIGS. 1 and 2 relate to the same components. Each reactor cell 5-7 comprises two copper mesh electrodes spaced by a distance of 1.65 cm, the copper mesh area of each electrode being 14×4.5=63 cm². Within each cell (between the copper electrodes) is an air-permeable fixed bed of glass beads (6 mm in diameter). For the purposes of the Examples, each plasma cell was individually powered by a high voltage, high frequency, neon sign transformer power supply. The input voltage of these power sources was controlled by a Variac (ZENITH Electric Company Ltd., Wavendon). The energy consumption (Variac+reactor cells) was measured by a Plug-in Power and Energy Monitor (Model 2000MU). The plug-in power and energy monitor did not work when the voltage was lower than 70 volts. We were therefore unable to locate the power monitor after the Variac and measure the power for each reactor cell. In the following Examples we therefore measured the total power consumption of Variac and the three reactor cells.

The apparatus illustrated in FIG. 5 was used for Example 5. The apparatus is similar to that shown in FIG. 2 and therefore like parts in the two Figures are depicted by the same reference numerals. The apparatus of FIG. 5 therefore comprises a housing 2 formed with an inlet 3 and an outlet 4. Located in series within the housing 2 are three reactor cells 5-7 and two catalyst beds 8 and 9 positioned such that gas entering the apparatus 1 through inlet 3 has to flow through each of the cells 5-7 and beds 8 and 9 before reaching outlet 4.

The cells 5-7 in the apparatus of FIG. 5 are identical with each other and are of the construction disclosed in WO-A-0014010. More specifically, each reactor cell 5-7 comprises two copper mesh electrodes spaced by a distance of 1.65 cm, the copper mesh area of each electrode being 14×4.5=63 cm². Within each cell (sandwiched between the copper electrodes) is a gas-permeable fixed bed of glass beads (6 mm in diameter). For the purposes of the Examples, each plasma cell was individually powered by a high voltage, high frequency, neon sign transformer power supply. The input voltage of these power sources was controlled by a Variac (ZENITH Electric Company Ltd., Wavendon).

Catalyst bed 8 incorporates a proprietary manganese dioxide catalyst (“Catalyst A”) supported on an aluminium honeycomb. Catalyst A is capable of decomposing ozone. Catalyst bed 9 incorporates a proprietary low temperature copper oxide/manganese dioxide oxidation catalyst (“Catalyst B”) capable of decomposing ozone and oxidising carbon monoxide.

The apparatus further comprises means (not shown) such as a fan or the like for moving gas through the apparatus from inlet 3 to outlet 4 via cells 5-7 and beds 8 and 9.

The apparatus of FIG. 6 was used for Example 8 and (with some modification) for Examples 4 and 5. All dimensions in FIG. 6a are in millimetres. The apparatus includes reactor cells 10-12 and a catalyst bed 13. The apparatus further incorporates FID detectors 14 and 15, the former being provided between reactor cell 12 and catalyst bed 13 and the latter being provided downstream of catalyst bed 13.

Each reactor cell 10-12 comprised two copper mesh electrodes with the copper mesh area of each electrode being 12×12=144 cm².

A modified version of this apparatus was used for Examples 4 and 5. The modification included removal of the catalyst bed 13 and omission of FID detector 15.

### EXAMPLE 1

This Example employed the apparatus of FIG. 3 for the removal of ethylene from a carrier gas comprised of a 4:1 mixture of nitrogen and oxygen. In the arrangement illustrated in FIG. 3, the “Plasma Reactor” was an apparatus as illustrated in FIG. 2.

Various experiments were conducted using a gas pressure of 1 bar, an ethylene concentration in air of 111.6 ppm and a total flow rate of 1 litre/min.

Experiments were conducted with power applied to all three cells (also depicted as A-C in FIG. 2) using primary input mains voltages as applied to the transformer of 25V and 30V. The output from the transformer had a frequency of 33 kHz. For the purposes of comparison, a further experiment was conducted using cell A only and a voltage of 25V.

The results are shown in Table 1.

TABLE 1

| | | | | N₂O | NOx |
|---|---|---|---|---|---|
| Input | | Ethylene | Ozone | concen- | Concen- |
| voltage | Reactor cell | destruction | concentration | tration | tration |
| (V) | configuration | (%) | (ppm) | (ppm) | (ppm) |
| 25 | A | 1.3 | 18.1 | 0 | <1 |
| 25 | A + B + C | 44 | 195.2 | 3.4 | <1 |
| 30 | A + B + C | 95 | 1389.9 | 41.1 | <1 |

It can be seen from the above table that, with a voltage of 25V, the use of all three cells A-C resulted in 44% destruction of ethylene in comparison with only 1.3% destruction with cell A alone was used. Ethylene destruction increased to 95% when all three cells A-C were used with a voltage of 30V although there was an increase in the concentrations of ozone and N₂O generated without any significant production of the oxides of nitrogen (NOx).

### EXAMPLE 2

The apparatus illustrated in FIG. 2 was employed for measuring the destruction of toluene in an experimental set-up as depicted in FIG. 4.

This Example was conducted using a carrier gas comprising a mixture of 80% nitrogen and 20% oxygen and containing 110 ppm of toluene. The gas pressure was 1 bar and the total flow rate through the reactor was 1 litre/min.

The input voltages used were as shown in Table 2. The output from the transformer was 13.4 kV (pk-pk) with a frequency of 39-43 kHz.

The peak at 2880 cm⁻¹ in the FTIR spectrum of toluene was used as a reference for calculating toluene concentration.

An experiment was conducted using an input voltage of 109.8V applied to all of cells A-C. For the purposes of comparison, cell A alone was used with an input voltage of 112.0V.

The results are shown in Table 2.

TABLE 2

| | Input | Energy | | | | Plasma | Toluene |
|---|---|---|---|---|---|---|---|
| Input | Power | density (J/ | | | β | cell | destruction |
| voltage (V) | (W) | Litre) | X₀ (ppm) | X (ppm) | (J/Litre) | configuration | (%) |
| 112.0 | 13.4 | 804 | 110 | 109.9 | 8 × 10⁵ | A | 0.1 |
| 109.8 | 41.7 | 2502 | 110 | 30.8 | 2000 | A + B + C | 72 |

Notes:
1. X/Xo = exp(−E/β)
Beta (β) = (−E)/In (X/Xo)
X: Toluene concentration after reaction (ppm);
Xo: Initial concentration of Toluene (ppm);
E: Energy density (J/litre);
Beta (β): Represents the energy density required for bringing down the concentration of toluene to 1/e of its initial concentration.

The three cell arrangement significantly reduces the 0 value indicating a significant enhancement of the energy efficiency of the process. This equates to a factor of 400 for 3 cells in series compared to a single cell.

It will be seen that simultaneous operation of all three cells A-C in the apparatus resulted in 72% destruction of toluene. In contrast, there was virtually no destruction of toluene when only cell A was operated.

### EXAMPLE 3

This Example monitors production of ozone and N₂O in an apparatus of the type shown in FIG. 2.

Experiments were conducted using a gas pressure of 1 bar and a total airflow rate of 1 litre/min. Investigations were conducted using different voltages and combinations of “activated” cells (i.e. cells to which power was applied).

The results are shown in Table 3 which show enhanced ozone generation for air when using multiple plasma cells in series compared to one cell, with no detectable NOx production.

TABLE 3

| Input | | Ozone | N₂O | NOx |
|---|---|---|---|---|
| voltage | Plasma cell | concentration | concentration | concentration |
| (V) | configuration | (ppm) | (ppm) | (ppm) |
| 25 | A | 3.9 | 0.2 | <1 |
| 25 | A + B + C | 62.7 | 0.7 | <1 |
| 30 | A + B + C | 1432.5 | 33.3 | <1 |
| 35 | A + B + C | 1732.5 | 78.3 | <1 |

The input voltage to the transformer is as shown in Table 3. The output frequency was 33 kHz.

### EXAMPLE 4

This Example was conducted using a modified version of the apparatus of FIG. 6 for the removal of toluene from air. The modification involved removal of the catalyst bed 13 and downstream FID detector 15 from the apparatus of FIG. 6. The resulting apparatus was, in effect, a scaled-up version of the apparatus shown in FIG. 2.

Each plasma cell was powered by a High Voltage High Frequency neon sign transformer PSU with the input voltage of the PSU being controlled by a Variac.

Toluene concentration in the air stream both before and after plasma treatment was measured by industrial FID detector 14.

Ozone and NOx concentrations in the airflow after plasma treatment was measured using a Gastec pump and test tubes. The detection limit for NOx (NO₂+NO) was 0.01 ppm.

Air flow through the apparatus was about 300 litres per minute which equated to an air velocity at the surface of each plasma cell of 0.4 m s⁻¹.

The input concentration of toluene was 25 ppm in the air flow.

Separate experiments were conducted with power applied to the first cell (A), the first and second cells (A+B) and all three cells (A+B+C). The input voltage was 55 V.

The results are shown in Table 4.

TABLE 4
Toluene Destruction

| | Toluene | | | Air | Deposited |
|---|---|---|---|---|---|
| Cell | Conversion | Input | Plug-in | velocity | Energy |
| (140 * 140 mm) | (%) | (V) | (Watt) | (m/s) | (J/Litre) |
| A | 13 | 55 | 30 | 0.4 | 6 |
| A + B | 45 | 55 | 60 | 0.4 | 12 |
| A + B + C | 100 | 55 | 92 | 0.4 | 18.4 |

The figures in the final column of Table 4 demonstrate the improved energy density values obtained using the scaled-up apparatus compared to the smaller scale unit in Table 2.

A further series of experiments was conduced using the apparatus to measure ozone and NOx (NO+NO₂) generated by the apparatus. For the purpose of this series of experiments, all three cells A+B+C were powered, the air flow rate through the apparatus was about 300 litres per minute and there was no toluene in the input air stream. The results are shown in Table 5.

TABLE 5
Ozone and NOx (NO + NO₂) Measurement

| Cell | | NOx | Input | Plug-in | Air velocity |
|---|---|---|---|---|---|
| (140 * 140 mm) | Ozone (ppm) | (ppm) | (V) | (Watt) | (m/s) |
| A + B + C | 80 | <0.01 | 55 | 90 | 0.4 |
| A + B + C | 120 | <0.01 | 70 | 130 | 0.4 |

The results in Table 5 demonstrate that there was no (less than 0.01 ppm) NOx after three plasma cells.

The experiment for which the results are shown in Table 5 was repeated but with 50 ppm toluene in the input air stream. The results (not shown) were the same as those in Table 5 thus indicating that the concentration of toluene (0 ppm and 50 ppm) in the input air stream does not influence ozone and NOx formation.

### EXAMPLE 5

The apparatus employed in Example 4 was used, with all three cells A+B+C powered, for the destruction of toluene at input levels of 10 ppm, 25 ppm and 50 ppm with destruction at each input levels being measured at Deposited Energy values of 16, 19.5, 23 and 29 J 1⁻¹.

Air flow through the apparatus was about 300 litres per minute giving a face velocity (through the plasma reactors) of about 0.4 m s⁻¹.

The results are shown in FIG. 7.

The results demonstrate that the three cell system of this design enables high level VOC removal at commercially viable flows and powers.

### EXAMPLE 6

This Example employed the apparatus of FIG. 5 (and modifications thereof) in an experimental set up as depicted in FIG. 4 in order to conduct a series of experiments investigating decomposition at ambient temperature of toluene in a carrier gas system comprised of a 4:1 mixture of nitrogen and oxygen (representing air).

For all experiments, the carrier gas was maintained at a pressure of 1 bar with a flow rate of 10 SLM. The toluene was introduced into the carrier gas flow by allowing a certain amount of nitrogen (controlled by a mass flow controller) to pass through a bubbler containing toluene kept in a water bath at room temperature (293K).

The degree of decomposition of the toluene and the identity of the products were determined by FTIR spectroscopy using a long-path gas cell and an FTIR spectrometer with a resolution of 1 cm⁻¹. The concentration of toluene (measured at 2880 cm⁴) was determined by using the standard reference spectra of QASoft-Infrared Analysis, Inc. The concentrations of O₃ (1052 cm⁻¹), CO (2116 cm⁻¹), CO₂ (2362 cm⁻¹) and N₂O (2235 cm⁻¹) were calculated according to their standard spectra from QASOFT.

The experiments identified in the following Table 6 as (i)-(vii) were conducted in accordance with the apparatus configurations/conditions listed in the third column of the table. For convenience, the middle column of the table also gives a descriptive “short name” for each experiment to facilitate an understanding of the results. For experiments (i)-(v) a mixture of carrier gas and toluene was introduced into the housing 2 via the inlet 3. Experiments (vi) and (vii) used an alternative arrangement as described in Table 1. The condition “plasma on” was effected by applying to the power supplies of each reactor cell 5-7 an input voltage of 45V and an input electric power of 57 W. The condition “plasma off” means there was no voltage/power input to the reactor cells 5-7.

TABLE 6

| Expt | | |
|---|---|---|
| No. | “Short Name” | Configuration/Condition |
| (i) | “No Plasma” | Apparatus as in FIG. 5, plasma off. |
| (ii) | “Plasma Alone” | Catalyst beds 8 and 9 removed, |
| | | plasma on. |
| (iii) | “Plasma + Catalyst A” | Catalyst bed 9 removed, plasma on. |
| (iv) | “Plasma + Catalyst B” | Catalyst bed 8 removed, plasma on. |
| (v) | “Plasma + Catalyst A + | Apparatus as in FIG. 5, plasma on |
| | Catalyst B” | |
| (vi) | “Ozone + Toluene” | The carrier gas (without toluene) was |
| | | passed through the apparatus with |
| | | plasma on but with catalyst beds 8 and |
| | | 9 removed. 70 ppm of toluene as then |
| | | introduced into housing 1 downstream |
| | | of reactor cell 7 and upstream of the |
| | | vacant catalyst position via an inlet (not |
| | | shown). |
| (vii) | “Ozone + Toluene + | As for (vi) but with catalyst bed 8 |
| | Catalyst A” | in place. |

The results are shown in Table 7.

TABLE 7
Concentration (ppm) from outlet 4

| | Toluene | CO | CO₂ | O₃ | N₂O | NOₓ |
|---|---|---|---|---|---|---|
| (i) No Plasma | 70 (0%) | 0 | 0 | 0 | 0 | ¹nd |
| (ii) Plasma alone | 45 (36%) | 16 | 19 | 1327 | 23 | nd |
| (iii) Plasma + Catalyst A | 0 (100%) | 48 | 80.1 | 0 | 18 | nd |
| (iv) Plasma + Catalyst B | 0 (100%) | 8 | 72 | 117 | 21 | nd |
| (v) Plasma + Catalyst | 0 (100%) | 10 | 110 | 0 | 17.5 | nd |
| A + Catalyst B | | | | | | |
| (vi) Ozone + Toluene | 70 (0%) | 0 | 0 | 766 | 12 | nd |
| (vii) Ozone + Toluene + | 0 (100%) | 16 | 25 | 0 | 13 | nd |
| Catalyst A² | | | | | | |

¹nd—none detected by FTIR - therefore concentration <1 ppm.
²Similar results are obtained using catalyst B in place of catalyst A and for catalysts A and B together

The figures in parenthesis in the above Table represent percentage destruction of toluene.

It can be seen from the results in Table 7 that experiment (i) in which the toluene/carrier gas mixture was passed through the reactor cells 5-7 (but without a non-equilibrium plasma being generated) and through the catalyst beds 8 and 9 did not result in any decomposition of toluene since the outlet concentration was 70 ppm (i.e. equal to the inlet concentration). Furthermore, experiment (ii), which was also comparative, in which the toluene/carrier gas mixture was passed through reactor cells 5-7 with generation of a non-equilibrium plasma therein (but without the catalyst beds 8 and 9 in position) resulted in destruction of 25 ppm of toluene (representing 36% destruction) with production of the specified amounts of CO and CO₂ resulting from toluene oxidation. A significant amount (1327 ppm) of ozone was also produced.

In contrast, experiment (iii), which is in accordance with the invention, in which the toluene/carrier gas mixture was passed through the reactor cells 5-7 (with non-equilibrium plasma being generated therein) and through catalyst bed 8 (containing the ozone destruction catalyst) resulted in complete destruction of toluene and ozone with production of higher amounts of CO and CO₂ than experiment (ii). There was no detectable production of NOₓ gases.

A comparison of the results of experiments (ii) and (iii) (where the latter utilised the ozone decomposition catalyst and the former did not) demonstrates the significant enhancement of toluene destruction achieved by the use of the ozone decomposition catalyst in accordance with the invention.

Experiment (iv) demonstrates that catalyst B was effective for complete removal of toluene and reduced levels of CO (indicating more complete oxidation of toluene) but there was some ozone (117 ppm) in the discharged gas from outlet 4.

Experiment (v) demonstrates that the combination of catalysts A and B resulted in complete destruction of toluene, complete destruction of ozone and production of elevated levels (as compared to the use of catalysts A or B alone) of carbon dioxide, thus indicating enhanced oxidation of toluene.

As a significant observation, none of experiments (ii)-(v) (in which a non-equilibrium plasma was generated in the reactor cells 5-7) resulted in the production of detectable amounts of NOₓ. As a general observation, the experiments did result in the production of the specified amounts of N₂O which is a known phenomenon in packed-bed plasma discharges.

Reference is now made to experiments (vi) and (vii). In experiment (vi)—which is comparative—the carrier gas (without toluene) was passed through reactor cells 5-7 in which a non-equilibrium plasma was generated but catalyst beds 8 and 9 were omitted. It will be seen that there was no destruction of toluene but, as expected, there was significant production of ozone. Experiment (vii)—which is in accordance with the invention, was carried out in a similar manner to experiment (vi) save that catalyst bed 8 was included in the apparatus. It can be seen from the results in Table 7 that all toluene was destroyed and all ozone decomposed. Production of CO₂ was less than in the case of experiments (iii)-(v) but nevertheless experiment (vii) demonstrates that combining plasma treated air with toluene and passage of the mixture over a catalyst capable of decomposing ozone does result in decomposition of the toluene.

### EXAMPLE 7

Example 6 was repeated but using a concentration of 88 ppm cyclohexane in the carrier gas instead of 70 ppm toluene and a total flow rate of air of 1.0 litre/min.

Experiments (i)-(iii), (vi) and (vii) were carried out as for Example 6 using (where appropriate) an input voltage of 32V and input electrical power of 10-12 W to generate the non-equilibrium plasma.

The results are shown in Table 8.

TABLE 8
Concentration (ppm) from outlet 4

| | Cyclohexane | CO | CO₂ | O₃ | N₂O | NO_{X} |
|---|---|---|---|---|---|---|
| (i) No Plasma | 88 (0%) | 0 | 0 | 0 | 0 | nd |
| (ii) Plasma alone | 67 (24%) | 11 | 16 | 1143 | 18 | nd |
| (iii) Plasma + Catalyst | 0 (100%) | 37.3 | 102 | 0 | 21 | nd |
| A | | | | | | |
| (vi) Ozone + | 88 (0%) | 0 | 1.9 | 985 | 16 | nd |
| Cyclohexane | | | | | | |
| (vii) Ozone + | 0 (100%) | 22 | 68 | 0 | 24 | nd |
| Cyclohexane + | | | | | | |
| Catalyst A | | | | | | |

The figures in parenthesis in the above Table represent percentage destruction of cyclohexane.

As can be seen from Table 8, experiment (i) (in which the cyclohexane was passed through reactor cells 5-7 without plasma generation and then through catalyst beds 8 and 9) did not result in any decomposition of cyclohexane. Experiment (ii) (in which the cyclohexane was passed through reactor cells 5-7 with plasma generation but not through catalyst beds 8 and 9) resulted in about 24% decomposition of cyclohexane with significant ozone production.

In contrast, experiment (iii) which is in accordance with the invention resulted in complete decomposition of the cyclohexane with production of significant quantities of its decomposition products (i.e. CO and CO₂). All ozone generated by the reactor cells 5-7 was decomposed by the catalyst bed 8.

As in the case of Example 6, experiments (vi) and (vii) demonstrates that admixture of plasma treated air with (in this case) cyclohexane and passage of the mixture over a catalyst capable of decomposing ozone results in complete decomposition of the cyclohexane.

### EXAMPLE 8

This Example employed the scaled-up apparatus of FIG. 6 which included, downstream of the final plasma reactor, a catalyst bed comprising a copper oxide/manganese dioxide oxidation catalyst capable of decomposing ozone and oxidising carbon monoxide.

Three runs were conducted each using an inlet air stream to the apparatus containing 71 ppm toluene. Air flow through the apparatus was about 300 litres per minute.

In a first run, the three plasma cells were retained in the apparatus but no power was applied to the cells. Therefore this run determined the effect of the catalyst only.

In a second run, the three plasma cells were all powered but the catalyst was removed and this run therefore demonstrated the ability of the plasma cells alone to destroy toluene.

In a third run, all three plasma cells were powered and the catalyst bed was in position. This run therefore demonstrated the combined effect of the plasma cells and the catalyst.

The results are shown in Table 9, for which measurements of toluene output were determined when the value had become constant (after about 4 hours).

TABLE 9
Influence of Catalyst and the Combination of
Catalyst and Plasma on Toluene Removal

| Cell | Toluene | Toluene | | | Plug- | Air |
|---|---|---|---|---|---|---|
| (140 * 140 | Input | output | Conversion | Input | in | velocity |
| mm) | (ppm) | (ppm) | (%) | (V) | (Watt) | (m/s) |
| Catalyst only | 71 | 71 | 0 | | | 0.4 |
| Plasma (3 | 71 | 41 | 42 | 70 | 125 | 0.4 |
| cells) | | | | | | |
| Plasma (3 | 73 | 27.5 | 62 | 70 | 125 | 0.4 |
| cells) + | | | | | | |
| Catalyst | | | | | | |

It will be seen from the results in Table 9 that the catalyst itself (i.e. without powering of the plasma cells) did not result in removal of any toluene. Operation of the three cells (but with the catalyst removed) resulted in a toluene conversion of 42%. When the catalyst was used in combination with all three plasma cells being operational then a conversion of 62% of toluene was achieved.

Reference is now made to FIG. 8 which is a plot of toluene concentration in the air output stream of the apparatus versus time for each of the three runs in Table 9.

For the first run in Table 9 (i.e. with catalyst only) no toluene was detected in the output stream during the first one hour of the run due to adsorption of toluene by the catalyst. There were then increasing amounts of toluene in the output steam until such time as the catalyst became saturated (after about four hours). Subsequently the amount of toluene in the output stream was 71 ppm (i.e. equivalent to the amount in the input stream).

For the second run in Table 9 (conducted with all three plasma cells operational but with the catalyst bed removed) the amount of toluene in the output stream was 42 ppm from the beginning of the run since there was no adsorption by the plasma cells and the catalyst bed (which is capable of adsorbing toluene) was not in position.

For the third run in Table 9 (conducted with all three plasma cells operational and the catalyst bed in position) no toluene was detected in the output stream during the first one hour of the run due to adsorption by the catalyst bed. The amount of toluene in the output stream then increased until the catalyst bed became saturated after about four hours, subsequent to which the amount of toluene in the output stream was 27.5 ppm.

Although the combination of the three operational plasma cells and catalyst did not reduce the toluene concentration to 0, the apparatus could be located upstream of a conventional activated carbon based VOC adsorption system which affects final removal of the toluene. The combination of the apparatus of the invention with an activated carbon based VOC adsorption system means that the consumption of activated carbon in the latter can be reduced.

1. Gas treatment apparatus comprising a gas-flow path and a plurality of reactor units through which gas may flow arranged in series along said path, said reactor units being adapted to generate a non-equilibrium plasma.

2. Apparatus as claimed in claim 1 comprising at least three of said reactor units arranged in series.

3. Apparatus as claimed in claim 1 wherein the reactor units are reactor cells comprising:
(i) a pair of spaced, air-permeable electrodes,
(ii) an air-permeable fixed bed of a dielectric material extending between the electrodes; and
(iii) means for applying a potential difference across the electrodes to generate a non-equilibrium plasma in the bed between the electrodes,
said cells being arranged such that the gas flow path is through the electrodes and the fixed beds.

4-6. (canceled)

7. Apparatus as claimed in claim 3 wherein dielectric material has a dielectric constant of less than 25.

8. Apparatus as claimed in claim 7 wherein the dielectric material is glass.

9. Apparatus as claimed in claim 3 wherein the air permeable fixed beds of the reactor cells comprise discrete bodies of the dielectric material in contacting relationship.

10. Apparatus as claimed in claim 9 wherein said bodies comprise beads.

11. Apparatus as claimed in claim 10 wherein said beads have a diameter of 1 to 12 mm.

12. Apparatus as claimed in claim 1 wherein provided downstream of the last reactor unit in series along said gas flow path is at least one catalyst bed incorporating a catalyst capable of decomposing ozone.

13. Apparatus as claimed in claim 12 wherein the ozone decomposition catalyst is manganese dioxide.

14. Apparatus as claimed in claim 12 additionally comprising a catalyst capable of oxidising carbon monoxide admixed with, or located downstream of, the catalyst capable of decomposing ozone.

15. Apparatus as claimed in claim 14 wherein the catalyst capable of oxidising carbon monoxide comprises copper oxide.

16. (canceled)

17. A method of treating gas to remove gas-borne contaminants therefrom, the method comprising passing the gas to be treated in series through a plurality of reactor units in which a non-equilibrium plasma is generated.

18. (canceled)

19. (canceled)

20. A method according to claim 16 wherein the reactor units are reactor cells comprising:
(i) a pair of spaced, air-permeable electrodes; and
(ii) an air-permeable fixed bed of a dielectric material extending between the electrodes,
said cells being arranged such that the gas flow path is through the electrodes and the fixed beds and said non-equilibrium plasma being generated by application of a potential difference to the electrodes of a cell.

21-23. (canceled)

24. A method as claimed in claim 20 wherein dielectric material has a dielectric constant of less than 25.

25. A method as claimed in claim 24 wherein the dielectric material is glass.

26. A method as claimed in claim 20 wherein the air permeable fixed beds of the reactor cells comprise discrete bodies of the dielectric material in contacting relationship.

27. A method as claimed in claim 26 wherein said bodies comprise beads.

28. A method as claimed in claim 27 wherein said beads have a diameter of Ito 12 mm.

29. A method as claimed in claim 16 wherein provided downstream of the last reactor cell in series is a catalyst bed incorporating a catalyst capable of decomposing ozone.

30. A method as claimed in claim 29 wherein the ozone decomposition catalyst is manganese dioxide.

31. A method as claimed in claim 29 wherein additionally provided downstream of the last reactor unit in series is a catalyst capable of oxidising carbon monoxide admixed with, or located downstream of, the catalyst capable of decomposing ozone.

32. A method as claimed in claim 31 wherein the catalyst capable of oxidising carbon monoxide comprises copper oxide.

33. Apparatus for decomposing a pollutant material dispersed in a gas, the apparatus comprising a gas flow path along which are provided for gas flow therethrough.
(i) at least one reactor unit which is adapted to generate a non-equilibrium plasma and produce ozone in the gas, and
(ii) downstream of (i), at least one catalyst bed incorporating a catalyst capable of decomposing ozone.

34-55. (canceled)

56. A method as claimed in claim 17 wherein the gas is air.
